(19) United States
(12) Patent Application Publication
(10) Pub. No.: US 2015/0093363 A1
(43) Pub. Date: Apr. 2, 2015

(54) OSTEOGENIC DIFFERENTIATION OF MESENCHYMAL STEM CELLS

(71) Applicant: BIOMATCELL AB, Göteborg (SE)

(72) Inventors: Karin Ekström, Kallered (SE); Peter Thomsen, Vastra Frolunda (SE); Jukka Lausmaa, Goteborg (SE); Omar Omar, Goteborg (SE); Xiaoqin Wang, Goteborg (SE)

(21) Appl. No.: 14/398,896

(22) PCT Filed: May 10, 2013
(86) PCT No.: PCT/SE2013/050526

§ 371 Date: Nov. 4, 2014

(30) Foreign Application Priority Data
May 10, 2012 (SE) 1250477-5

(51) Int. Cl. C12N 5/077; A61K 35/32

(52) U.S. Cl. CPC C12N 5/0654; A61K 35/32; C12N 2506/1384; C12N 2502/1157; C12N 2501/052
USPC 424/93.7; 435/377

(57) ABSTRACT

The present invention relates to a method for inducing and/or promoting osteogenic differentiation using extracellular vesicles and the use thereof.

### FIELD OF INVENTION

The present invention relates to a method of inducing or promoting differentiation of cells, extracellular vesicles for use in inducing or promoting said differentiation, the use of said vesicles and a method of treatment using said vesicles.

### INTRODUCTION

The mechanisms of early bone formation at implant surfaces or at the site of injury and the factors influencing the maintenance of bone-implant contact, stability and function are not fully understood. An increased knowledge of the pathways whereby inflammatory cells and stem cells and progenitor cells communicate at the surface of the implant is important for the understanding of how the next generation of implants for clinical use should be optimized. With greater knowledge, in the future, we will hopefully be able to produce new and better implants for improved osseointegration or better drugs for bone healing.

The monocyte/macrophage system plays a central role in host defense, wound healing and immune regulation at biomaterial surfaces. Monocytes and mesenchymal stem cells rapidly migrate to implanted material surfaces and are localized in close proximity to each other, prior to extracellular matrix deposition and bone formation. It has been shown that conditioned medium from human monocytes, containing e.g. proinflammatory cytokines, promote the osteogenic differentiation of human mesenchymal stem cells (hMSCs). How the monocytes communicate with the MSCs is not fully determined, although it has been suggested that they communicate in absence of direct cell-to-cell contact.

Extracellular vesicles (EV) including exosomes play an important role in cell-to-cell communication. A suggested general mechanism of cell-to-cell communication relates to a delivery of RNA by transfer through exosomes, probably occurring in the microenvironment but potentially also at distance. Exosomes are small membrane vesicles (40-100 nm) of endocytic origin which are released into the extracellular milieu upon fusion of multivesicular bodies with the plasma membrane. Exosomes provide a mode of communication between cells, where one cell can release exosomes that can influence other cells in the microenvironment or over a distance.

Exosomes are released from many cells and their functions depend on the cellular origin and the condition for the producing cells which give them their characteristic composition. For example, exosomes originating from cells exposed to oxidative stress was shown to convey protective messages against stress in recipient cells.

However little is known about if and how EV's influence the differentiation of the recipient cells.

### SUMMARY OF INVENTION

The object of the present invention is to provide a method for inducing and/or promoting osteogenic differentiation of cells, preferably mesenchymal stem cells, more preferably human mesenchymal stem cells (hMSC). The invention also relates to the use of extracellular vesicles to increase bone regeneration and to favour osseointegration of implants.

In a first aspect the present invention relates to a method of inducing and/or promoting osteogenic differentiation of target stem cells comprising:
a. providing conditioned medium from cell culture of non-target cells or extracellular vesicles isolated from non-target cells; and
b. adding the medium or the extracellular vesicles to the target stem cells.

In a second aspect the present invention relates to isolated extracellular vesicles for use in osteogenic differentiation of target stem cells.

In a third aspect the present invention relates to a medium comprising extracellular vesicles obtained by non-target cells for use in osteogenic differentiation of target stem cells.

In a fourth aspect the present invention relates to an implant surface comprising a coating exposing extracellular vesicles.

In a fifth aspect the present invention relates to an implant surface comprising a coating of immobilized stimulated monocytes, macrophages or mesenchymal stem cells that are capable of producing exosomes.

In a sixth aspect the present invention relates to a method of treating a patient comprising collecting blood or tissue sample from the patient, isolating non-target cells, culturing and stimulating the non-target cells, isolating exosomes produced by the non-target cells and administrating the exosomes to the patient.

In a seventh aspect the present invention relates to the use of the isolated extracellular vesicles for the treatment of bone damages, osteoporosis, osteogenesis or in bone fixation.

In an eight aspect the present invention relates composition comprising isolated extracellular vesicles for the treatment of bone damages, bone voids, osteoporosis or osteogenesis.

In an ninth aspect the present invention relates to a bone void filler comprising isolated extracellular vesicles.

### BRIEF DESCRIPTION OF FIGURES

FIG. 1. Transmission electron microscopy picture of exosomes isolated from the conditioned medium of the human monocyte cell line HMC-1, and immunogold labelled against CD63.

FIG. 2. Flow cytometric analysis of monocyte derived exosomes conjugated to anti-CD63 latex beads. Exosomes were immunostained against the tetraspanins CD9, CD63 and CD81 (right panel) and isotype matched controls (left panel).

FIG. 3. Western blot analysis of proteins extracted from monocyte exosomes and their donor cells.

FIG. 4. Detection of exosomal and cellular total and small RNA from monocytes. Electropherograms disclosing size distribution in nucleotides (nt) and fluorescence intensity (FU) of total RNA in (a) monocyte exosomes and cells (b) and small RNA in exosomes (c) and cells (d).

FIG. 5. Flow cytometric analysis (similar as for the monocyte exosomes) was applied for detection of exosomes from mesenchymal stem cells. The data shows that the mesenchymal stem cells release exosomes that are positive for CD9, CD63 and CD81.

FIG. 6. Nanoparticle tracking analysis of MSC exosomes.

FIG. 7. Transmission electron microscopy picture of MSC exosomes immunogold labeled against CD63 (bar 20 nm).

FIG. 8. Electropherograms disclosing size distribution in nucleotides (nt) and fluorescence intensity (FU) of total RNA in MSCs and exosomes isolated from MSC cultures.

FIG. 9. Transfer of MSC exosomes to monocytes MSC exosomes were isolated, labelled with a green fluorescent dye (PKH67, SIGMA) and added to monocyte in cultures. The uptake of the labelled vesicles was analyzed after 24 h by (a) flow cytometer and (b) fluorescence microscope. Control; PKH67 stained PBS. DAPI was used for nucleus staining. Green cells are monocytes that are positive for the green exosomes, showing they have taken up exosomes or exosomes attached to their surface.

FIG. 10. Transfer of MSC exosomes to MSCs similar as in FIG. 9, MSC exosomes were isolated, labelled with a green fluorescent dye (PKH67, SIGMA) and added to MSC cultures. The uptake was analyzed after 24 h by (a) flow cytometer and (b) fluorescence microscope. Control; PKH67 stained PBS. DAPI was used for nucleus staining. Green cells are MSCs that are positive for the green exosomes, showing they have taken up exosomes or exosomes attached to their surface.

FIG. 11. Exosomes released from monocytes/macrophages stimulated with LPS were isolated, labelled with a green fluorescent dye (PKH67, SIGMA) and added to MSCs in culture. MSCs were cultured in the presence of green exosomes for ˜72 h, after which cells were analyzed by flow cytometer (a) in the fluorescence microscope (b). DAPI was used for nucleus staining. Green cells are MSCs that are positive for the green exosomes, showing they have taken up exosomes or exosomes attached to their surface. Exosomes from monocytes/macrophages are taken up/attach to mesenchymal stem cells.

FIG. 12. Gene expression of Runx2 and BMP-2 in hMSCs cultured, for 72 h, in unconditioned control medium (ctrl), in monocyte conditioned medium (CM) or in medium supplemented with exosomes (exo).

### DETAILED DESCRIPTION OF THE INVENTION

In the present invention the term “target stem cell” or “target stem cells” means a cell or cells that are to be induced to osteogenic differentiation by conditioned medium or extra cellular vesicles from non-target cells.

The term “conditioned medium” means the medium used for cell culture after the cultured cells have been removed.

The signalling to cells is generally regarded as an effect of molecules which are presented in a soluble form and/or on a surface, being the plasma membrane of cells or matrix-associated. The present invention relates to cell-cell communication mediated by cell-derived, around 20-400 nm, or 50-200 nm, extracellular vesicles, containing for example proteins, mRNAs and microRNAs. The vesicles attach to, fuse or are internalized by the target cells and exert a regulatory effect in target cells. This communication may induce and/or promote osteogenic differentiation of cells such as stem cells, especially human mesenchymal stem cells.

The present inventors have developed a method of inducing and/or promoting osteogenic differentiation of target cells. The method comprises providing conditioned medium from a cell culture of non-target cells or isolated extracellular vesicles released from non-target cells. In one embodiment the extracellular vesicles are exosomes. The extra cellular vesicles are provided by culturing the non-target cells, optionally during stimulation of the non-target cells to release said vesicles, and isolation of the conditioned medium or the vesicles. The non-target cells may be cultured for various times for example 1 hour or more, or 1 day or more, or 2 days or more, or 3 days or more. The medium or the vesicles are added to or brought into contact with the target cells which may be stem cells. The vesicles are then taken up by or attached to the target cells inducing and/or promoting osteogenic differentiation.

The target cells may be any suitable cell type or cell line but may also be progenitor cells or stem cells. The cells may for example be osteoblasts, osteoclasts, mast cells, muscle cells, fat cells or mesenchymal stem cells (MSCs) such as human MSCs.

To the inventors' knowledge, there are no publications describing the cross-talk between cells such as inflammatory cells and target cells such as mesenchymal stem cells via extracellular exosomes or other extracellular vesicles with similar properties to induce and/or promote osteogenic differentiation. The inventors have found that cells such as inflammatory cells send messages to other cells such as mesenchymal stem cells resulting in increased expression of bone differentiation genes in the recipient cells, for example stem cells. These messages are exosomes and/or other extracellular vesicles.

The induction, and/or promotion, of osteogenic differentiation of the target stem cells according to the present invention may be performed by stimulating cells, other than the target cells, herein called non-target cells. These stimulated non-target cells could be any suitable cells and can for example be monocytes, macrophages, erythrocytes, osteoclasts, mast cells, myoblasts, keratinocytes, adipocytes or any other inflammatory cells or non-inflammatory cells and stem cells for example mesenchymal stem cells. Preferably the non-target cells are monocytes, macrophages or stem cells, and in a preferred embodiment these are human monocytes, macrophages or stem cells for example hMSC. The method can be performed both in vivo and in vitro.

Without being bound by theory, the phenotype of the target or non-target cells may play a role in the success of the induction and/or promotion of osteogenic differentiation. It is known that EVs from non-target cells with different phenotypes also have different phenotypes and functions. Furthermore, the phenotype of the target cells may influence whether the EVs can bind to and deliver their message to the target cells, and further whether the target cell can be directed in a specific direction. In one embodiment of the present invention the non-target cells are autologous or non-autologous. In another embodiment the target cells are autologous or non-autologous. The benefit of using autologous may be the limited immunological response while non-autologous cells from a healthy person, or a person not suffering from the disease to be treated, may be beneficial when it comes to the regeneration or healing process.

The cells could be stimulated in a variety of ways for example by the use of a stimulating agent such as lipopolysaccharides (LPS), cytokines, chemokines or any other stimuli or combinations thereof. The amount of stimulating agent may be in the range of 1 to 100 ng/ml, for example 1 ng/ml or more, or 5 ng/ml or more, or 10 ng/ml or more, or 20 ng/ml or more, or 100 ng/ml or less, or 70 ng/ml or less, or 50 ng/ml or less, or 30 ng/ml or less. In one embodiment the concentration range of stimulating agent is 1 to 20 ng/ml, in another embodiment the concentration is 5 to 50 ng/ml, and in yet another embodiment the concentration is 5 to 15 ng/ml.

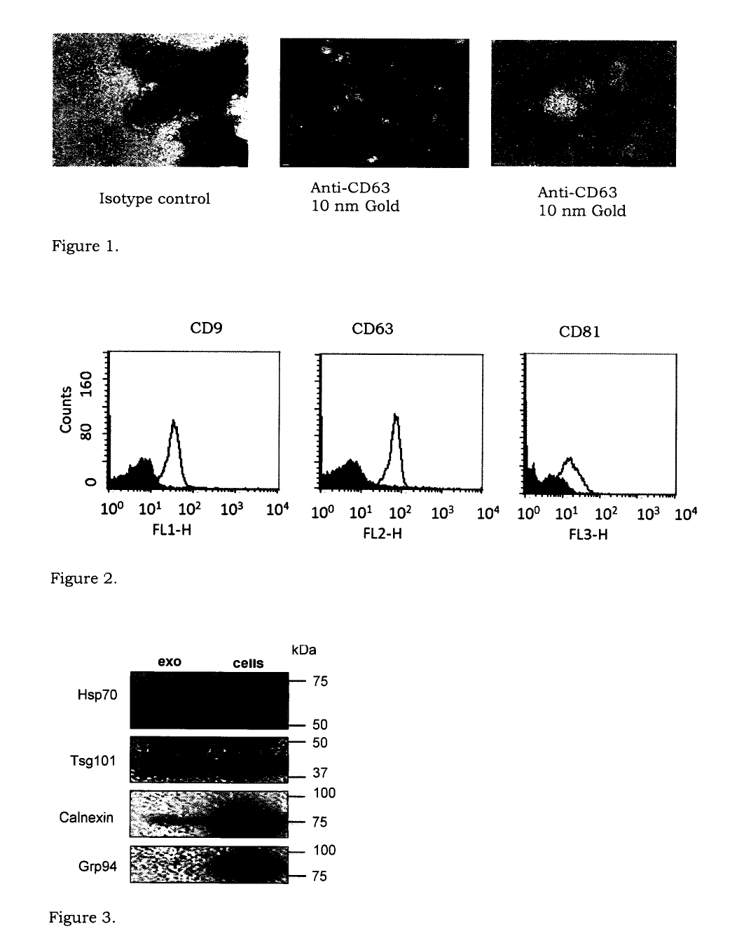

Without being bound by theory, it is believed that the non-target cells, especially the stimulated non-target cells, produce differentiation stimulating extra cellular vesicles, small sacs of membrane released from a cell, for example exosomes, which when in contact with or taken up by the target stem cells induce osteogenic differentiation. These vesicles, inducing and/or promoting osteogenic differentiating vesicles, could be isolated from conditioned medium from anyone of the non-target cells, for example monocytes, macrophages, erythrocytes, osteoclasts, mast cells, adipocytes, and stem cells, for example mesenchymal stem cells. The vesicles could be mixed with other biomolecules such as growth factors. The size of the extracellular vesicles may be in the range of 20 to 400 nm for example 20 nm or more, or 50 nm or more, or 80 nm or more or 100 nm or more, or 400 nm or less, or 300 nm or less, or 250 nm or less, or 200 nm or less, or 150 nm or less. FIG. 1 discloses exosomes isolated from conditioned medium.

In one embodiment the isolated EVs are a combination of EVs released from two or more different cell types. In another embodiment the isolated EV's are a combination of EVs released from cells stimulated using two or more different types of stimulating agents. In yet another embodiment the isolated EVs are a combination of EVs released from two or more different cell types wherein each cell type is stimulated using at least one different type of stimulating agent.

The cytokines, growth factors or other signal substances or combination of substances which are responsible for the differentiation is not yet fully determined. The cytokines, growth factors and other signal substances may also be used to stimulate the non-targeting cells to release extracellular vesicles, for example with a specific phenotype and/or function. Other signal substances can be for example hormones. Potential cytokines are IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, 11-19, IL-20, IL-21, IL-22, IL-23, IL-24, IL-25, IL-26, IL-27, IL-28, IL-29, IL-30, IL-31, IL-32, IL-33, IL-34, IL-35 or IFN-types, or combinations thereof. Potential growth factors are BMP, TGF, VEGF, TNF or FGF or combinations thereof. Examples of chemokines would be any one of the types CC, CXC, CX3C and XC such as CCL 2, CCL 3, CCL 5, CCL 7, CCL 8, CCL11, CCL 13, CCL 17, CCL 22, CCL24, CCL26, CCR1, CCR2, CCR3, CCR4, CCR5 or combinations thereof. Other signal substances can be for example hormones.

The vesicles may further comprise other substances such as other proteins, growth factors (BMP, TGF, VEGF, TNF or FGF or combinations thereof), mRNA, miRNA or siRNA or other small regulatory RNA.

In one embodiment the non-targeting cells are stimulated using lipopolysaccharides (LPS), cytokines, chemokines, growth factors (BMP, TGF, VEGF, TNF or FGF or combinations thereof) or any other stimuli or combinations thereof to release extra cellular vesicles containing proteins, growth factors (BMP, TGF, VEGF, TNF or FGF or combinations thereof), mRNA, miRNA or siRNA or other small regulatory RNA or combinations thereof.

In yet another embodiment the non-targeting cells are stimulated using lipopolysaccharides (LPS), cytokines, chemokines, growth factors (BMP, TGF, VEGF, TNF or FGF or combinations thereof) or any other stimuli or combinations thereof to release extra cellular vesicles containing proteins, growth factors (BMP, TGF, VEGF, TNF or FGF or combinations thereof), mRNA, miRNA or siRNA or other small regulatory RNA or combinations thereof. These vesicles are then added to or brought into contact with the target cells in combination with lipopolysaccharides (LPS), cytokines, chemokines, growth factors or any other stimuli or combinations thereof.

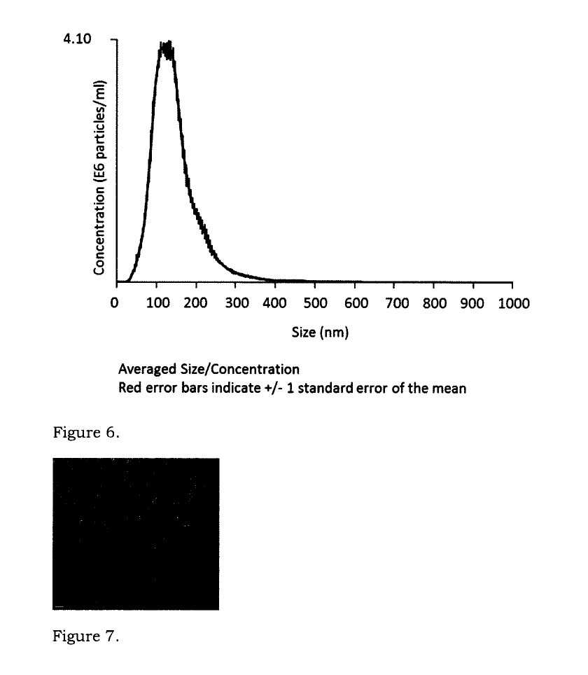

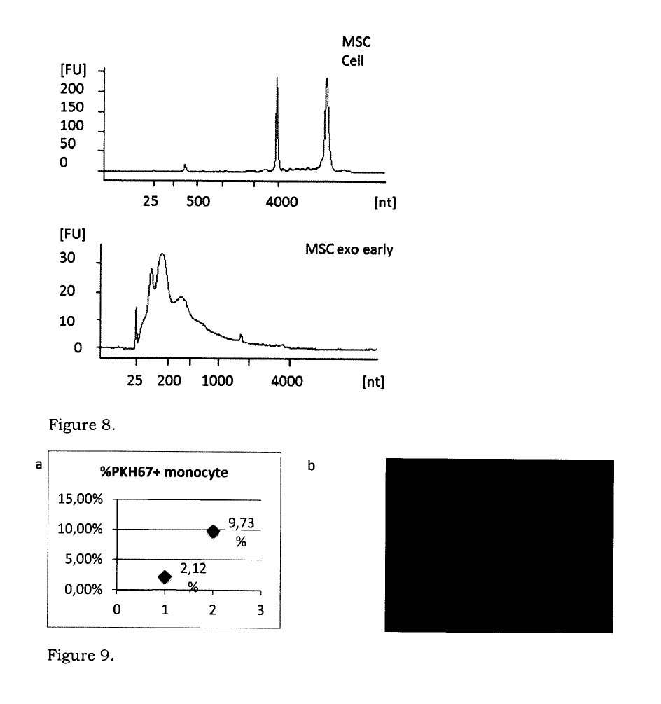

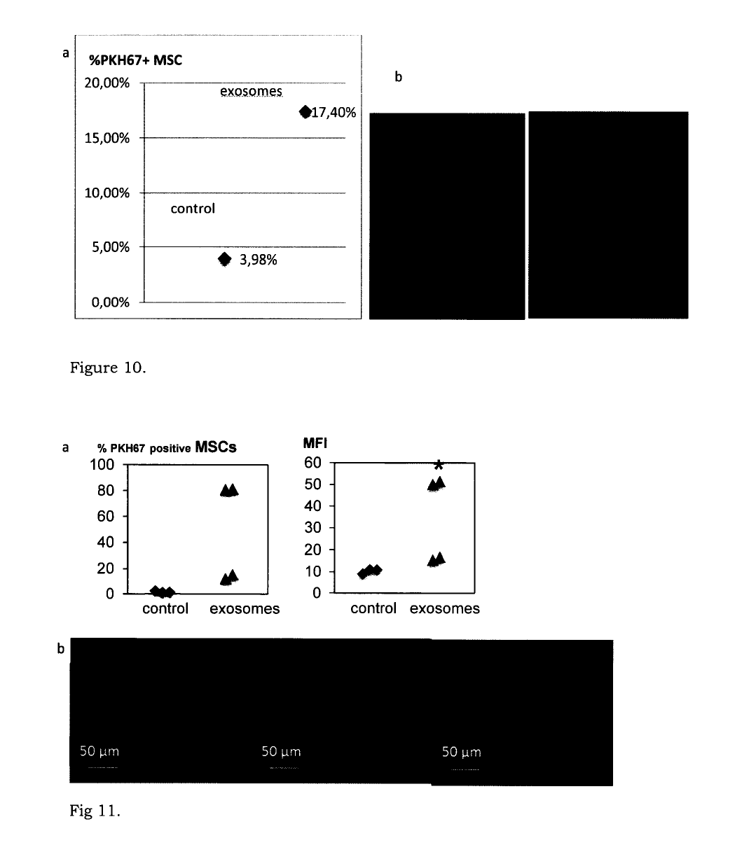

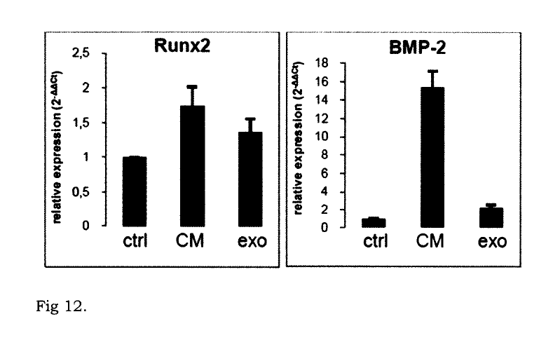

The present inventors have shown that both MSCs and monocytes release extracellular vesicles containing RNA, FIGS. 1-8, and the inventors have also shown that target MSCs take up the released extracellular vesicles, FIG. 9-10. The inventors also showed that the osteogenic differentiation was promoted when hMSCs were treated according to the present invention, as exemplified by increased gene expression of RUNX2 and BMP-2, FIG. 12.

The extracellular vesicles could be mixed with a cell culture medium (or condition medium) such as a PBS buffer or any other saline buffer or any other medium. The medium may contain vesicles from more than one type of non-target cell for example two, three or four cell types. For example the medium may contain a mixture of vesicles from monocytes and hMSC, or monocytes, hMSC and macrophages. The extracellular vesicles may be provided to the target cells both in vitro and in vivo.

The conditioned medium from cell culture of non-target or the isolated extracellular vesicles can be added to a site where bone regeneration is needed. The conditioned medium or the vesicles can also be delivered together with the target cells. By delivering the medium or the vesicles to a site where the target cells are located the osteogenic differentiation will be induced. The delivery can be done by injection or through implantation of a delivery vehicle. Extracellular vesicles such as exosomes have the benefit of causing moderate or no immunological response in comparison with for example synthetic liposomes.

The extracellular vesicles are added to or brought into contact with the target cells in an amount sufficient enough to induce and/or promote osteogenic differentiation.

Furthermore, the vesicles may be provided to the stem cells by coating or immobilizing the vesicles on a surface, for example on an implant surface, or as a part of a drug delivery system. The implant surface could be a metal surface such as titanium or titanium oxide, or a ceramic surface such as a calcium phosphate surface. The drug delivery system could for example be a hydrogel or a biodegradable material which would slowly release the vesicles. The hydrogel could for example be hyaluronic acid or chitosan or polyvinyl alcohol or a combination of the same. In another embodiment, an implant surface is coated or immobilized with cells for in vivo release of extracellular vesicles. For example, a surface may be coated or immobilized with stimulated monocytes, macrophages or mesenchymal stem cells which would release an increased number of exosomes in order to induce and/or promote osteogenic differentiation at the site of the implant.

The method according to the present invention may also be used to treat a patient. The method comprises
collecting blood or tissue sample from a human, for example the patient himself, using any available suitable technique;
isolating non-target cells and culture the cells and optionally stimulate the non-target cells to produce inducing and/or promoting extracellular vesicles;
isolating said extracellular vesicles produced by the non-target cells; and
administering the extracellular vesicles to the patient.

The administration may be performed systemically or locally by any suitable technique for example by a syringe to the location at which osteogenic differentiation is needed or wanted.

The present invention can be used as a supplementary treatment during bone surgery, implant surgery, bone healing treatment or bone or teeth fixation treatments. The isolated extracellular vesicles can be immobilized on various scaffolds and implants such as implants for dental applications such as teeth, hip joints or knees or any other bone implant. The vesicles can also be immobilized on bone fixation implants such as screws or fixation plates. Furthermore, the present invention can be used to treat various bone related diseases and damages such as bone void filling material, osteophytes, craniosynostosis, osteoarthritis, various osteoitis diseases, osteopetrosis, osteopenia osteoporosis or osteogenesis.

### EXAMPLES

### Example 1

### General Description of the Process

Isolation and Culture of Monocytes:

Monocytes are isolated from human blood using magnetic separation and cultured on different biomaterials and with or without stimulation (e.g LPS). Mesenchymal stem cells are obtained from human bone marrow by gradient separation. After 72 h, cells are harvested and exosomes isolated from the conditioned medium.

Exosome Isolation and Detection:

Exosomes will be isolated using a method based on repeated centrifugation and filtration steps to remove cell debris, apoptotic bodies etc. followed by ultracentrifugation to pellet the exosomes. Alternative isolation methods may also be used. Exosomes will be detected using a combination of methods including electron microscopy and detection of a number of markers often found on exosomes (e.g CD9, CD63, CD81, Tsg101) and markers that should be absent in exosomes (Calnexin) using flow cytometry and Western blot.

The mRNA and microRNA Content of Exosomes:

Microarrays will be performed on exosomes from monocytes and MSCs exposed to different stimulation to evaluate the mRNA and microRNA content.

Uptake Experiments:

Isolated exosomes will be labelled with a fluorescent dye, added to MSCs in culture and the uptake analysed after different time point using fluorescence microscopy and flow cytometry.

Evaluation of Osteogenesis:

Histological staining (von Kossa) and markers for bone formation (osteocalcin, runx2, collagen type I) is evaluated using RT-PCR.

### Materials & Methods

Human monocytes were obtained from buffy coats by magnetic separation (purity 90-95%, n=4). The monocytes were treated with LPS (10 ng/ml) for 72 h and the conditioned medium (CM) was collected. Human adipose-derived mesenchymal stem cells (MSCs) were cultured and the conditioned medium was collected. Exosomes were isolated from the CM by repeated centrifugation and filtration steps and detected using flow cytometry. For flow cytometric analysis, exosomes were conjugated to anti-CD63 latex beads and immunostained against the tetraspanins CD9, CD63 and CD81. Exosomes were also visualized using transmission electron microscopy and nanoparticle tracking analysis. RNA from the different types of vesicles and their donor cells was extracted and the size distribution pattern analyzed using a Bioanalyser. hMSCs were cultured in medium supplemented with monocyte exosomes, CM or control medium for 72 h. The osteogenic differentiation was evaluated using real-time PCR analysis (Runx2, BMP-2, n=4). The relative quantification of the target gene expression was calculated by the dd-Ct method. In separate experiments, human monocytes or hMSCs were cultured in medium supplemented with PKH67 stained vesicles and the uptake examined using flow cytometry and microscopy.

### Results

Flow cytometric analysis revealed that the LPS-stimulated monocytes and MSCs release exosomes positive for the tetraspanins CD9, CD63 and CD81, which are markers often used for exosome detection, see FIGS. 2 and 5.

FIG. 11 discloses that exosomes from monocytes/macrophages are taken up/attached to mesenchymal stem cells. MSCs cultured in the presence of green PKH67 stained exosomes are positive in FL-1 compared with the negative controls, indicating the exosomes attach to or fuse with MSCs or are internalized by the MSCs. Furthermore, culture of hMSCs in medium supplemented with PKH67 labeled green MSC exosomes show that MSCs are positive for the green dye, suggesting that MSCs communicate with other MSCs via exosomes, see FIG. 10. In addition, culture of monocytes with green MSC exosomes also revealed that a portion of the monocytes had taken up or internalized the green exosomes, see FIG. 9.

Culture of hMSCs in monocyte CM or in medium supplemented with pure exosomes isolated from the CM, for 72 h, resulted in significantly increased expression levels of Runx2 (fold change 1.7±0.3 and 1.4±0.2, respectively) and BMP-2 (fold change 15.4±1.7 and 2.3±0.3, respectively) compared to control medium, see FIG. 12.

The results demonstrate that LPS-stimulated human monocytes release factors that enhance osteogenic differentiation of mesenchymal stem cells and that at least a part of this effect is due to the communication via exosomes.

### Example 2

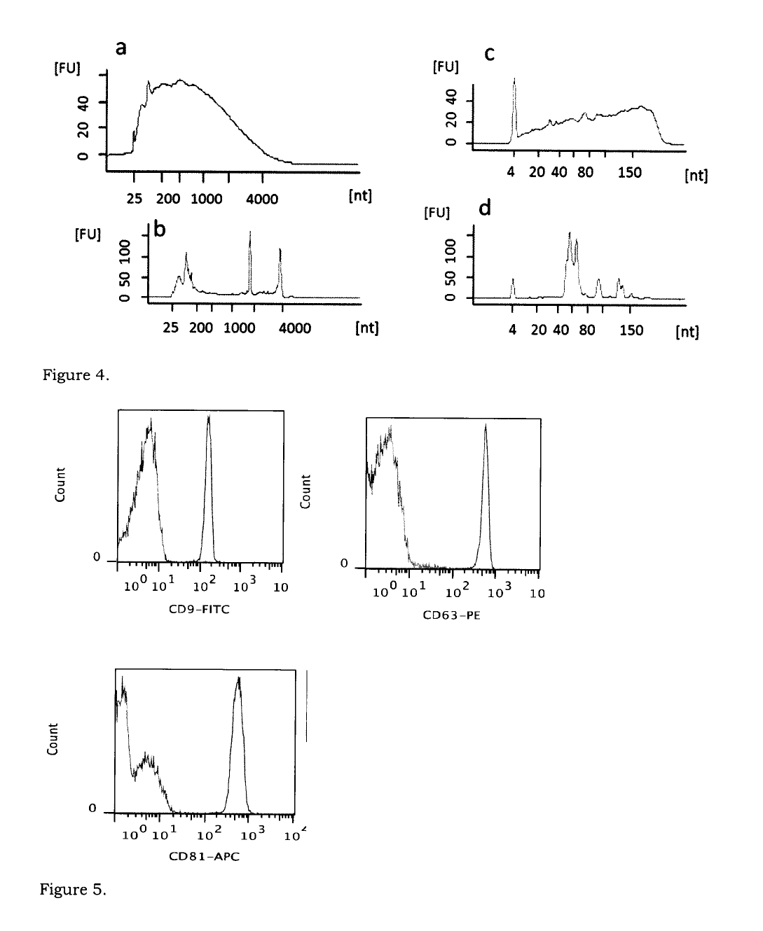

Human primary LPS stimulated monocytes. Exosomes were isolated after 3 day culture and RNA extracted. Bioanalyzer analysis of cellular and exosomal total and small RNA was performed. The electropherograms show, FIG. 4, the size distribution in nucleotides (nt) and fluorescence intensity (FU) of total RNA in (a) exosomes and cells (b) and small RNA in exosomes (c) and cells (d).

1. A method of inducing and/or promoting osteogenic differentiation of target cells comprising:
a. providing inducing and/or promoting osteogenic differentiating isolated extracellular vesicles released from non-target cells; and
b. adding the extracellular vesicles to the target cells.

2. The method of claim 1 wherein the non-target cells are stimulated monocytes, macrophages or mesenchymal stem cells or other inflammatory or non-inflammatory cells.

3. The method of claim 2 wherein the non-target cells are stimulated with lipopolysaccharides, cytokines, chemokines or any other stimuli.

4. The method of claim 2, wherein the non-target cells are stimulated or modified in any other way to release extracellular vesicles inducing and/or promoting osteogenic differentiation of mesenchymal stem cells.

5. The method of claim 1 wherein the extracellular vesicles are released from monocytes.

6. The method of claim 1 wherein the vesicles provided are a combination of vesicles released from two or more different types of cells, or a combination of vesicles released from cells stimulated using two or more different types of stimulating agents, or a combination of EV's released from two or more different cell types wherein each cell type is stimulated using at least one different type of stimulating agents.

7. The method of claim 1 wherein the extracellular vesicles are exosomes.

8. Use of isolated inducing and/or promoting osteogenic differentiating extracellular vesicles from non-target cells for osteogenic differentiation of target cells.

9. The vesicles according to claim 8 wherein the non-target cells are stimulated monocytes, macrophages or mesenchymal stem cells or other inflammatory or non-inflammatory cells.

10. The vesicles according to claim 9 wherein the non-target cells are stimulated with lipopolysaccharides, cytokines, chemokines or any other stimuli.

11. The vesicles according to claim 8 wherein the vesicles are exosomes.

12. The vesicles according to claim 8 wherein the vesicles are suspended in a medium.

13. An implant comprising a coating exposing inducing and/or promoting osteogenic differentiating extracellular vesicles.

14. An implant comprising a coating of immobilized stimulated monocytes, macrophages or mesenchymal stem cells that are capable of producing inducing and/or promoting osteogenic differentiating extracellular vesicles.

15. The implant according to claim 13 wherein the implant is an implant for dental applications, hip joints, knees, screws or fixations plates.

16. The use according to claim 8 for the treatment of damages, osteoporosis, osteogenesis or in bone fixation.

17. A composition comprising isolated inducing and/or promoting osteogenic differentiating extracellular vesicles for the treatment of bone damages, bone void filling material, osteophytes, craniosynostosis, osteoarthritis, various osteoitis diseases, osteoporosis or osteogenesis.

18. A bone void filler comprising isolated inducing and/or promoting osteogenic differentiating extracellular vesicles.

19. The bone void filler according to claim 18 further comprising targeting stem cells.

20. A method of treating a patient comprising collecting blood or tissue sample from the patient, isolating non-target cells, culturing and stimulating the non-target cells, isolating extracellular vesicles produced by the non-target cells and administrating the extracellular vesicles to the patient.
